(19) United States
(12) Patent Application Publication
(10) Pub. No.: US 2023/0230664 A1
(43) Pub. Date: Jul. 20, 2023

(54) AUTOMATED RANDOMIZATION VALIDATION FOR AN RTSM SYSTEM

(71) Applicant: 4G Clinical LLC, Wellesley, MA (US)

(72) Inventors: Edward Alan Tourtellotte, Dover, MA (US); Jean van der Vaeren, Grez-Doiceau (BE)

(73) Assignee: 4G Clinical LLC, Wellesley, MA (US)

(21) Appl. No.: 18/100,388

(22) Filed: Jan. 23, 2023

Related U.S. Application Data
(63) Continuation of application No. PCT/US2023/060840, filed on Jan. 18, 2023.
(60) Provisional application No. 63/300,589, filed on Jan. 18, 2022.

(51) Int. Cl. G16H 10/20; G16H 10/60

(52) U.S. Cl. CPC G16H 10/20; G16H 10/60

(57) ABSTRACT

A method for managing a clinical trial for a drug includes determining a randomized patient list and a randomized kit list for the clinical trial, automatically validating the randomized lists, and configuring an RTSM system based on the validated lists. Validating the randomized patient list may include verifying that the patient list includes a balance or spread above a balance or spread threshold and verifying that the list has a randomness above a predetermined randomness threshold. Validating the randomized kit list may include verifying that the kit list has a randomness above a predetermined randomness threshold.

### RELATED APPLICATIONS

This application claims the benefit under 35 U.S.C. §119(e) of U.S. Provisional Application Serial No. 63/300,589, filed on Jan. 18, 2022, the entire contents of which are incorporated herein by reference.

### BACKGROUND

This disclosure relates to automated validation of randomized lists in clinical trial management, and configuration of Randomization and Trial Supply Management (RTSM) systems based on such validated randomized lists.

### BRIEF DESCRIPTION OF THE DRAWINGS

FIG. 1 is a diagrammatic view of an example system for managing a pharmaceutical trial.

FIG. 2 is a flow chart illustrating an example method of configuring an RTSM system for a clinical trial.

FIG. 3 is a flow chart illustrating an example method of validating a randomized patient list.

FIG. 4 is a flow chart illustrating an example method of validating a randomized kit list.

FIG. 5 is a flow chart illustrating an example method of interpreting a clinical trial specification using natural language processing.

FIG. 6 is a diagrammatic view of a natural language processing-based trial specification interpreter.

FIG. 7 illustrates an example graphical display of a randomized patient list, which may be output to a user in connection with validating the randomized patient list.

FIG. 8 and FIG. 9 illustrate example graphical displays of a randomized kit list, which may be output to a user in connection with validating the randomized patient list

FIG. 10 is a diagrammatic view of an example embodiment of a user computing environment.

### DETAILED DESCRIPTION

Randomization and Trial Supply Management (RTSM) systems generally execute a trial supply plan that is based on randomized patient lists and randomized kit lists, both of which may be generated according to trial parameters provided by a sponsor of the trial. Validating those randomized lists through automated processes and computer simulations enables efficient configuration of an RTSM system with statistically-sound randomization that is more reliably confirmed that under known manual processes.

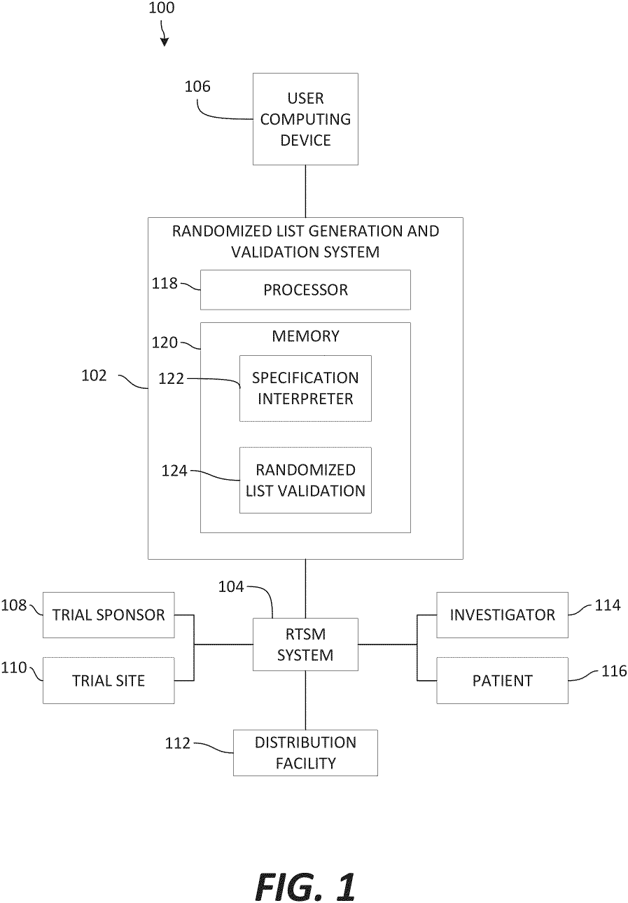

Referring now to the drawings, wherein like reference numerals represent the same or similar features in the various views, FIG. 1 is a diagrammatic view of an example system 100 for managing a clinical trial, such as a randomized pharmaceutical trial, for example. The system 100 may include a randomized list generation and validation system 102 and an RTSM system 104, either or both of which may be in electronic communication with a plurality of user computing devices 106 (one of which is shown in FIG. 1).

Generally, the randomized list generation and validation system 102 and the RTSM system 104 may be involved in different aspects of planning and administration for a pharmaceutical trial. As will be described in greater detail below, the RTSM system 104 may generally guide and administer the trial, whereas the randomized list generation and validation system 102 may generate randomized lists, including randomized patient lists and randomized kit lists, and validate the contents and randomness of those lists.

The RTSM system 104 may comprise one or more computing devices and may perform many operations for organizing and orchestrating a pharmaceutical trial, such as blinding, drug dispensing, and automatic drug resupply, in embodiments. The RTSM system 104 may be in electronic communication with one or more user computing devices 106 to receive input from the user (e.g., parameters of a trial) and to provide output to a user (e.g., a report on validated patient and kits lists, orders for one or more drugs, a status of a patient participating in the trial, information respective of a trial site, etc.).

The RTSM system 104 may be configured for communication and information exchange with a variety of different user types (that is, communication with one or more user computing devices respective of such user types). For example, in a given trial, the RTSM system 104 may provide an input/output interface for a clinical trial sponsor 108 (e.g., a pharmaceutical company). The trial sponsor 108 may input complete parameters of a clinical trial, or stage thereof, for the RTSM system 104, in embodiments. Such a complete set of parameters may include, for example, patient visits or other actions on patients, lots and operations on lots, inventory and operations on inventory at a site or depot level, shipments and operations on shipments, temperature excursions and their management, return of dispensed or non-dispensed drugs and related approvals and parameter, destruction status of inventory, actions on alerts or notifications, addition and parametrization of sites and depots, countries, setup of forecasting parameters such as a confidence dial and a long window dial, set up of site group enrollment rates, setup of users and their access rights, setup of cohorts, loading of kit and randomization lists, unblinding of a patient or a kit. The RTSM system 104 may then determine and output, to the trial sponsor, which resupply actions may have to be taken. The RTSM system 104 may also track and record the status of the trial based on data from one or more depots, sites, doctors, and patients, as will be described below, and may make such information available to the trial sponsor.

The RTSM system 104 may be in electronic communication with one or more trial sites 110, such as hospitals or clinics, for example. The RTSM system 104 may output instructions to a trial site 110, such as dispensing instructions for particular patients, for example. The RTSM system 104 may also receive data from a trial site 110, such as patient information, dispensing records, and the like, such that the RTSM system 104 has data indicative of which patients took which drugs involved in the trial and which sites, as when.

The RTSM system 104 may further be in communication with one or more distribution facilities 112, such as depots, warehouses, or other facilities involved in the distribution of, e.g., active drugs, placebos, and/or comparators involved in the trial to trial sites 110. The RTSM system 104 may issue shipping instructions to such a facility 112, for example. Such shipping instructions may be generated and/or transmitted automatically by the RTSM system 104, in embodiments, based on a supply plan. The RTSM system may also receive data from such facilities 112, such as shipping records and inventory information, such that the RTSM system 104 has data indicative of distributed inventories of drugs involved in the trial and remaining inventories, and the locations of those inventories. Drugs (e.g., active drug, comparator, and placebo kits) may be distributed and dispensed to patients according to the instructions of the RTSM system, and in particular according to the validated, randomized patient and kit lists generated according to this disclosure.

One or more investigators (e.g., doctors) 114 may also be in electronic communication with the RTSM system 104. The RTSM system 104 may output to such investigators 114 information about the drugs involved in the trial and information about the trial to enable the investigator to inform patients about the trial as appropriate. The RTSM system 104 may receive, from such investigators, enrollment information (e.g., the number of patients that the investigator has recommended for the trial, the characteristics of those patients, the specific patients that have enrolled, etc.), such that the RTSM system 104 has data indicative of enrollment quantities and characteristics of enrolled patients.

The randomized list generation and validation system 102 may include a processor 118 and a non-transitory, computer-readable memory 120 including instructions that, when executed by the processor 118, cause the processor 118 to perform one or more methods, algorithms, processes, etc. of this disclosure. The memory 120 may store one or more modules in the form of executable instructions (e.g., software) for execution by the processor 118. Example modules are described below.

The memory 120 may store a specification interpretation module 122, in embodiments. A user (e.g., via a user computing device which, as noted above, may be associated with a trial sponsor) may submit, and supply forecasting system 102 may receive, information indicative of parameters of a trial. The specification interpretation module 122 may process and parse the received information to extract and determine the trial parameters.

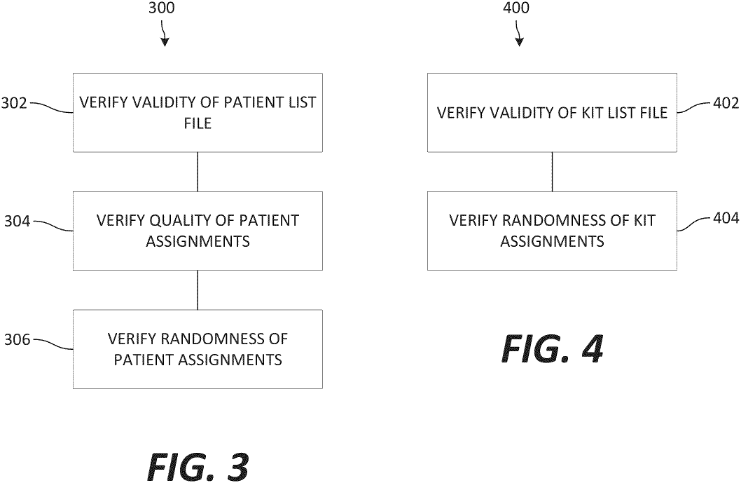

The memory 120 may also store a randomized list validation module 124 configured to automatically generate and validate randomized lists, such as randomized patient lists and randomized kit lists, based on the trial parameters determined by the specification interpreter 122. Example methods for automatically validating randomized lists will be described with respect to FIGS. 3 and 4.

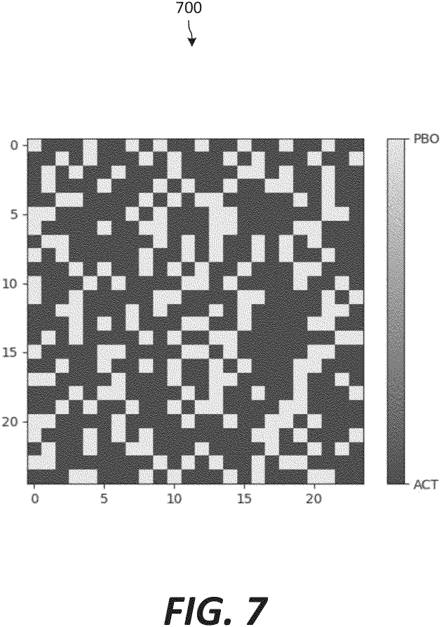

The randomized list generation and validation system 102 may provide an electronic user interface for use by the trial sponsor. For example, the validation system may output a validation report respective of one or more patient lists and/or kit lists. The validation report may include, for example, a graphical display of the patient list and/or kit list (an example of which is illustrated in and will be described with respect to FIG. 7), among other information.

One or both of the RTSM system 104 and the randomized list generation and validation system 102 may be implemented on a Software-as-a-Service (SaaS) basis, in an embodiment. Accordingly, the validation system and/or RTSM system 104 may be embodied in one or more servers, databases, etc., that are electronically accessible to users through the internet. Additionally or alternatively, one or both of the randomized list generation and validation system 102 and RTSM system 104, or one or more aspects or functions of the randomized list generation and validation system 102 and RTSM system 104, may be implemented on a local server for a trial sponsor, for example, or in local copies stored on user computing devices respective of the trial sponsor, trial sites, depots, etc.

Although the RTSM system 104 and the randomized list generation and validation system 102 are illustrated and described as separate systems, the RTSM system 104 and the randomized list generation and validation system 102, or some functionality thereof, may be implemented in a single system or in common computing resources, in embodiments.

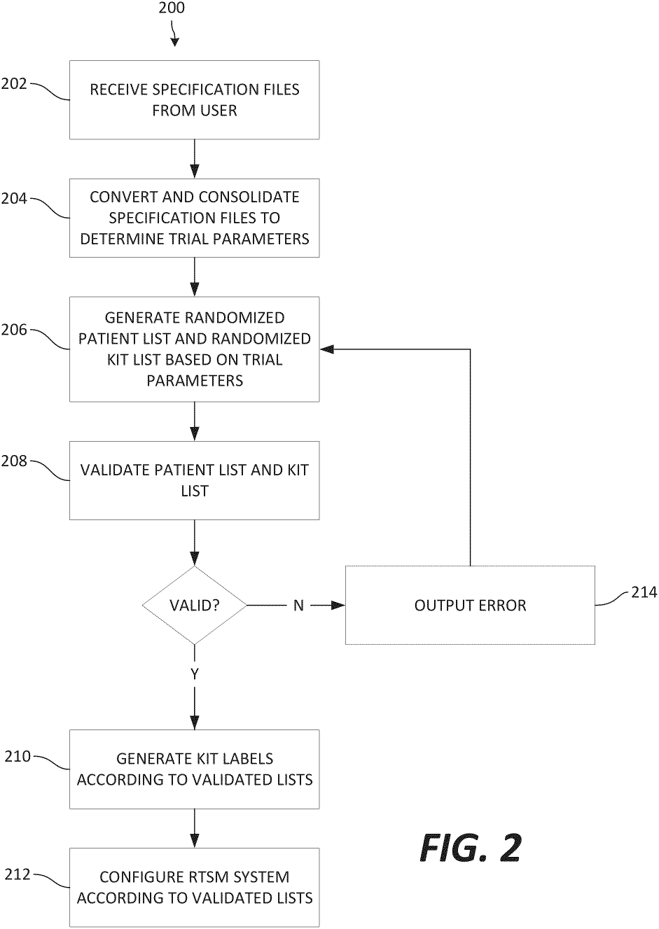

FIG. 2 is a flow chart illustrating an example method 200 of configuring an RTSM system with automatically validated randomized lists. The method 200, or one or more aspects of the method 200, may be executed by a combination of a validation system and an RTSM system, such as the randomized list generation and validation system 102 and RTSM system 104 of FIG. 1, for example.

The method 200 may include, at block 202, receiving one or more specification files from a user. The user may be, for example, a trial sponsor (e.g., a pharmaceutical company). The specification files may include one or more file types. For example, in an embodiment, the specification files may include one or more text files and one or more spreadsheet files. The text may be entered in free or natural form, rather than being responsive to a structured prompt, in embodiments. The specification files may include information available to the sponsor about a proposed pharmaceutical trial, such as block size, treatment arms, ratios, treatment kits, comparators and placebos, kit and sequence number ranges, cohorts, strata, geographical or demographic information, and the like.

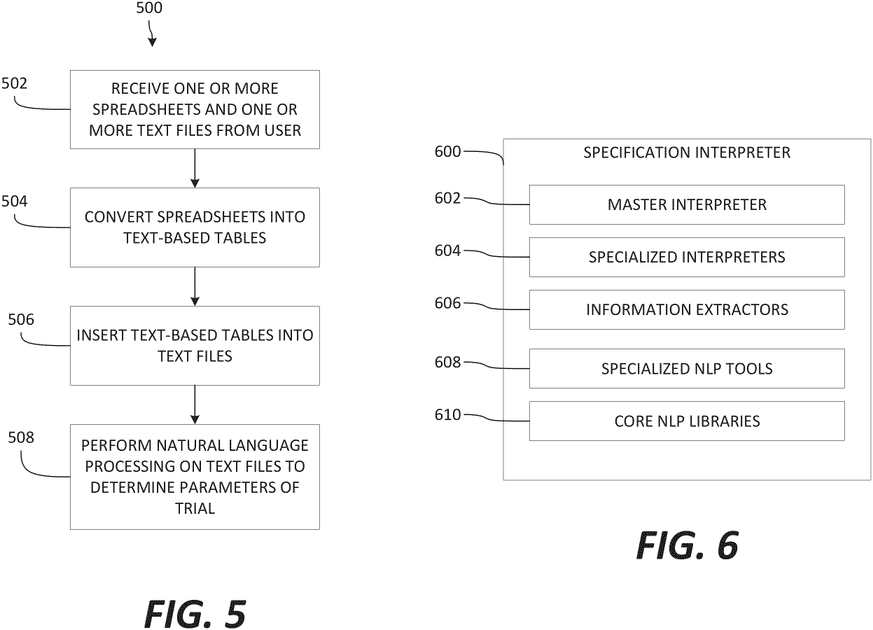

The method 200 may further include, at block 204, converting and consolidating the specification files to extract or otherwise determine parameters of a trial, including parameters of desired patient randomization lists and/or parameters of desired kit randomization lists, that are included in the specification files. An example method for performing block 204 will be described with respect to FIG. 5 and FIG. 6.

The method 200 may further include, at block 206, generating a randomized patient list and a randomized kit list based in the trial parameters determined at block 206. For example, the randomized patient list may have a randomized sequence of patients assigned to either an active drug arm or a placebo arm of the trial, or an active arm or comparator arm, or one of a number of different active drug strength arms. The randomized patient list may be based on individual randomization, in some embodiments (e.g., in which each patient has an equal likelihood as every other patient of being assigned to any given group, such as an active drug group, a placebo group, a comparator group, an active drug strength group, etc.). In other embodiments, the randomized patient list may be based on randomized blocks, and the blocks may have variable or random sizes, or another appropriate randomization technique may be employed. Similarly, the kit list may be individually randomized, or may be based on randomized blocks, and the blocks may have variable or random sizes, or another appropriate randomization technique may be employed.

The method 200 may further include, at block 208, validating the patient list and the kit list generated at block 206. Example operations for block 208 will be described with respect to FIGS. 3 and 4. Block 208 may be performed automatically upon generation of the patient list and kit list, in some embodiments.

Validating the patient list and the kit list at block 208 may include generating a report including validation results. For example, the report may include one or more graphical displays of the patient distribution and/or the kit distribution, as will be described with respect to FIGS. 7-9. The report may additionally or alternatively include information or tables respective of the patient list or kit list, and/or information respective of one or more simulations or mathematical analyses respective of the patient list or kit list, such as patient enrollment quantities and sequences in one or more simulations.

If the patient list or the kit list is not valid, the method 200 may output an error at block 212 and return to block 206 to generate a new randomized patient list and/or randomized kit list to replace the invalid list. The error output at block 212 may identify the validation step or portion that failed (e.g., one or more aspects of the methods 300, 400 of FIGS. 3 and 4). The error output at block 212 may include results of one or more simulations, such as one or more patient enrollment simulations, as well as an indication as to whether than simulation met or did not meet one or more thresholds regarding the randomized list.

If the patient list and the kit list are valid, the method 200 may include, at block 210, generating kit labels according to the validated lists. Block 210 may further include printing the kit labels and applying the kit labels according to the randomized list so that distribution and dispensing of kits may be effected based on the user’s randomization and other parameters.

If the patient list and the kit list are valid, the method 200 may include, at block 212, configuring an RTSM system according to the validated randomized patient list and validated randomized kit list, as well as according to the remaining trial parameters determined at block 206. The RTSM system may be configured to execute and monitor a trial plan that incorporates the randomized patient list and randomized kit list. Further, active drug, placebo, and/or comparator kits may be distributed and dispensed to patients according to the validated randomized patient list and the validated randomized kit list.

Due to the inclusion of automated document conversion and interpretration to determine trial parameters, automated generation of randomized lists, and automated validation of randomized lists, the system 100 and method 200 may enable automated configuration of an RTSM system for a particular clinical trial, requiring only freeform text and spreadsheet files from the initiator of the trial, without requiring intervening manual validation of randomized lists. Further, automated validation of randomized lists as set forth in the methods 200, 300, 400 provides more statistically-sound validation than current approaches, enabling more reliable trials.

FIG. 3 is a flow chart illustrating an example method 300 of automatically validating a randomized patient list. The method 300, or one or more portions thereof, may be performed by the randomized list generation and validation system 102 of FIG. 1.

The method 300 may include, at block 302, verifying the validity of the patient list file. Verifying the validity of the patient list file may generally include confirming that the list includes valid content such as, for example, that the randomized patient list matches or meets the user’s trial description. For example, block 302 may include verifying that arm ratios, block sizes, the quantity of blocks per strata, and other ratios and quantities in the patient list meet the trial description. Block 302 may include one or more of validating a total quantity of rows (i.e., patients) and a total quantity of blocks for the patient list, validating a quantity of rows and a quantity of blocks for one or more treatment arms, validating a quantity of rows and a quantity of blocks for one or more cohort and strata combinations, or validating a size and quantity of each of a plurality of block types.

The method 300 may further include, at block 304, verifying the quality of the patient assignments in the randomized patient list. Verifying the quality of the patient assignments may include, for example, verifying that the patient list includes an appropriate balance of active drug patients to placebo patients for the entire list and/or within each defined strata or cohort, or an appropriate balance of active drug patients to comparator drug patients, or an appropriate balance of patients receiving a first strength of the active drug to patients receiving a second strength of the active drug, and so on. In some embodiments, the balance (e.g., active drug patients as a percentage of all patients) within each grouping may be compared to a predetermined threshold and, if the balance is above the threshold or is within a certain range of the predetermined threshold, the balance may be considered valid. For example, if the trial aims to include ⅔ of patients in the active drug portion of the trial, simulations with between approximately 60% and approximately 72% of patients in an active drug treatment arm may be considered valid. The exact valid boundaries depend on several aspects of the clinical trial, such as the number of patients, stratifications, treatment efficacy, etc. In some embodiments, the balance of each strata or cohort may be calculated and, if the balance of any strata or cohort fails to meet the threshold, the entire patent list may be considered invalid. That is, if the balance of each strata or cohort meets the balance threshold, the patient list may have a valid balance.

Block 304 may include computing one or more simulations of patient enrollment, computing one or more balances for each simulation, and comparing the balance(s) of each simulation to the predetermined threshold, in some embodiments. In some simulation embodiments, each patient may have an equal chance of each of two or more blocking factors for the trial. In other simulation embodiments, different probabilities may be applied for different strata.

In embodiments in which a simulation is performed, a balance of the patient list may be calculated multiple times over the course of the simulation (e.g., after enrollment of 100 patients, then after enrollment of 200 patients, and so on), and the balance compared to a threshold each time. As a result, the evolution or progression of patient balance may be assessed over the course of the simulation. If the balance fails to meet the threshold at any of the calculated times in the simulation—i.e., if the number of patients in the treatment arm of the trial is too far above or too far below the desired ratio or percentage—the patient list may be considered invalid, in some embodiments. In some embodiments, the points in time of the simulation at which the balance is evaluated and/or enforced, including either the end of the simulated trial and/or one or more interim points in the trial, may be user-selected.

In addition to or instead of computing one or more balances of the patient list, a spread of the patient list may be calculated, in some embodiments, and the spread may be compared to a threshold. For example, a spread may be calculated for a patient list with multiple block types. In such embodiments, the quantity of treatment arm patients in each block type may be calculated, and the spread (e.g., standard deviation) of those quantities may be calculated and compared to a threshold. If the spread is below the threshold, the patient list may be valid at block 304.

Multiple separate patient lists may be generated from a trial specification, in some embodiments, and block 304 may be performed as to each of those patient lists, and the results of block 304 over multiple patient lists may indicate the quality of the trial specification parameters provided by the trial sponsor. That is, the results of the block 304 over multiple patient lists may indicate whether the trial parameters are compatible with desired patient randomization techniques (e.g., by virtue of the quantity or proportion of valid or invalid patient lists based on those parameters).

The method 300 may further include, at block 306, verifying the randomness of the patient assignments in the randomized patient list. For example, block 306 may include determining that block patterns are uniformly distributed throughout the patient list. In some embodiments, determining that block patterns are uniformly distributed may include determining a distribution of treatment patterns across blocks and computing a p-value for that distribution, which indicates the probability of obtaining a distribution at least as extreme as the subject distribution. The p-value may be computed using a chi-squared test, in some embodiments. In other embodiments, another appropriate mathematical approach for calculating a p-value. The p-value may be compared to a predetermined threshold (e.g., 0.05, or 5%), in some embodiments and, if the p-value exceeds the threshold, the list may be considered validly random.

In an example of determining a treatment pattern distribution, consider an example trial with a block size of three patients, with each block intended to include two active drug patients and one placebo patient. In this example, three treatment patters exist: AAP (i.e., the first and second patients in the block receive the active drug, and the third patient receives placebo); APA, and PAA. Thus, in this example, block 306 may include determining a quantity of blocks having each of these three patterns and calculating a p-value for the distribution of those block/pattern quantities.

In the method 300, if the patient list is determined to be invalid at any of blocks 302, 304, 306, then the patient list may be considered invalid. Conversely, if the patient list is determined to be valid at all blocks 302, 304, 306, the patient list may be considered valid.

FIG. 4 is a flow chart illustrating an example method of automatically validating a randomized kit list. The method 400, or one or more portions thereof, may be performed by the randomized list generation and validation system 102 of FIG. 1.

The method 400 may include, at block 402, verifying the validity of the kit list file. Verifying the validity of the kit list file may generally include confirming that the list includes valid content such as, for example, that the randomized kit list matches or meets the user’s trial description. For example, block 402 may include one or more of validating a total quantity of rows and a total quantity of blocks in the kit list, comparing the use (or nonuse) of kit blocks to user requirements regarding the use (or nonuse) of kit blocks, or validating a quantity of rows and a quantity of blocks for one or more kit types. In some embodiments, each section of a kit list may contain different kit types and use a different block size (e.g., as if each section was its own kit list). Accordingly, in some embodiments, block 402 may include evaluating the kit types and block sizes of each of a plurality of sections to ensure the kit types, block types, blocks count, and sequence range found for each section match the expectation.

The method 400 may further include, at block 404, verifying the randomness of the kit assignments in the randomized kit list. For example, block 404 may include calculating an average sequence of each kit type and confirming that kit types in the same section have an average sequence that is close to the middle of the section. Block 304 may further include, for non-blocked lists, calculating a distribution of run lengths (i.e., consecutive kit numbers) and comparing the distribution to an expected number of runs of each length. For example, block 406 may include determining that run lengths are uniformly distributed. In some embodiments, determining that run lengths are uniformly distributed may including calculating a p-value of the run length distribution. The p-value may be computed using a chi-squared test, in some embodiments. The p-value may be compared to a predetermined threshold (e.g., 0.05, or 5%), in some embodiments and, if the p-value exceeds the threshold, the list may be considered validly random.

In some embodiments, block 404 may include, for blocked kit lists, determining that block patterns are uniformly distributed throughout the kit list. In some embodiments, determining that block patterns are uniformly distributed may include determining a distribution of kit patterns across blocks and computing a p-value for that distribution, which indicates the probability of obtaining a distribution at least as extreme as the subject distribution. The p-value may be computed using a chi-squared test, in some embodiments. In other embodiments, another appropriate mathematical approach for calculating a p-value. The p-value may be compared to a predetermined threshold (e.g., 0.05, or 5%), in some embodiments and, if the p-value exceeds the threshold, the list may be considered validly random.

FIG. 5 is a flow chart illustrating an example method 500 of determining parameters of a proposed pharmaceutical trial, including patient, kit, and randomization parameters. The method 500, or one or more parts of the method 500, may be performed by the RTSM system 104, by the randomized list generation and validation system 102, and/or by another system in communication with the RTSM system, such as a forecasting system, in embodiments.

In some embodiments, portions of the method 500 related only to randomization parameters may be performed by the randomized list generation and validation system 102, independently of other trial parameters, in a separate process from that performed for determining the other parameters of the trial. For example, the randomized list generation and validation system 102 may be implemented as a standalone system from the RTSM system 104, to build randomized patent and/or kit lists separately from building out the remaining aspects of the trial. Accordingly, although the discussion below will address randomization parameters in conjunction with other trial parameters, it should be understood that aspects related to randomization may be performed alone.

The method 500 may include, at block 502, receiving a spreadsheet file and one or more text files from the user. The files may include the information about a study to be conducted that is available to the user at the time. The text files may be HTML files, for example. The spreadsheet and text files may be received via a file upload through a website provided by the supply forecasting system, for example. In embodiments, the supply forecasting system may provide an interface in which a user may enter freeform text to describe the proposed study (which entered freeform text may be or may become the one or more text files of block 502), and further upload one or more spreadsheets through the interface.

The method 500 may further include, at block 504, converting the spreadsheet into one or more text-based tables. The spreadsheet may be converted into one or more HTML-based tables, for example, or into whatever format the text-based files were provided in, in embodiments.

The method 500 may further include, at block 506, inserting the text-based tables into the one or more text files, and at block 508, performing natural language processing on the one or more text files to determine the parameters of the trial. Examples of the natural language processing that may be performed at block 508 will be described with respect to FIG. 6. Further detail on natural language processing is provided in US 2017/0154168 entitled “Generating RTSM Systems for Clinical Trials” by inventor Edward A. Tourtellotte and assigned to 4G Clinical, LLC, which application is hereby incorporated by reference in its entirety. Through the use of natural language processing (NLP), users can describe their trial in plain English, independently from the level of detail of information available, and the system can determine the available details of the trial for further processing.

FIG. 6 is a diagrammatic view of an example electronic specification interpreter 600 that applies natural language processing. The specification interpreter 600 may perform one or more aspects of the method 500 of FIG. 5, and in particular block 508, in embodiments. The specification interpreter 600 may include a master interpreter 602, one or more specialized interpreters 604, one or more information extractors 606, one or more specialized natural language processing (NLP) tools 608, and one or more core NLP libraries 610. The specification interpreter 600 can further include additional components, in embodiments.

A core NLP library, such as the core NLP libraries 610, can include information of a natural language (e.g., English), such as vocabulary and syntax, corpora, taggers, tokenizers, stemmers, lemmatizers, learning algorithms and more. The core NLP library 610 can be used by the specification interpreter 600 to examine the specification entered by a specification writer. For example, the specification interpreter 600 can rely on the core NLP library 610 to recognize words and stems, flag incorrect spelling, analyze grammar and meaning, detect logical operations and data definitions, and/or suggest alternative terms. In some situations, the core NLP library 610 can be provided by a third party, such as from certain programming language (e.g., Java or Python) development toolkits (e.g., Natural Language Tool Kit or NLTK).

A specialized NLP tool, such as the specialized NLP tools 608, can provide system-specific extensions to the core NLP library 610. For example, the specialized NLP tools 608 can include additional information for words, phrases, terms, syntax, reserved words/phrases, keywords, and operators, for example as may be commonly used in the context of certain fields (e.g., clinical trials). The specialized NLP tool 608 can also include advanced language analysis functionality, such as tokenizing, stemming, grammar, collocations, language corpus, sentence structure and meaning analysis, among other natural language processing features. The specialized NLP tools 608 can also include artificial neural networks.

The specialized NLP tools 608 can also be used by the specification interpreter 600 to examine the specification entered by a specification writer. For example, the specification interpreter 600 can rely on the specialized NLP tools 608 to recognize system-specific words, flag incorrect spelling, and/or suggest alternative terms. The specialized NLP tools 608 can be provided by a third party or internally developed. An information extractor, such as the information extractors 606, can extract certain information from the specification. One type of information extracted can include matrices (e.g., information structured in a tabular manner). A matrix may be used in specification development when it is more convenient to organize data in matrix form than in natural language. For example, a matrix can be used to define patient visit schedules and associated dosing regimens or other visit events; a matrix can also be used to define patient kit types, supply chain relationships and lead times, or any other data which is naturally defined in a matrix.

Another type of information extracted can include certain style of sentences such as definitions. A definition could for example be structured as [SUBJECT OF DEFINITION] [VERB PROPOSITION] [ITEM or LIST OF ITEMS], but structure could differ or be made more flexible to enable use of free English style in writing and yet extracting relevant information. Other styles of sentences can be defined as necessary.

The master interpreter 602 can receive a trial specification. The master interpreter 602 can perform top level analysis of the specification and can organize interpretation in a logical sequence of themes. A theme can include one or more defined trial topics, which can run across one or more sections of the specification document. A particular theme of the interpretation can correspond to a specialized interpreter 604, configured to analyze information relevant to the particular theme across the specification document (in one or more sections). The master interpreter 602 can invoke different specialized interpreters to process different themes of the trial through the specification.

The specialized interpreters 604 can include a study level interpreter, a randomization interpreter, an action interpreter, a drug supply interpreter, a manufacturing interpreter, an enrollment interpreter or other interpreters as required. A study level interpreter can interpret the high-level information in the specification. For example, high level information can include certain study level information, such as the total number of patients desired to be screened and randomized, the randomization scheme and method, and other required parameters. A randomization interpreter can interpret randomization information in the specification. The randomization interpreter may recognize a size of the rand list, treatment arms used, treatment arm ratios, stratification and cohorts, strata assignment to each portion of the list (e.g., rand numbers [101, 200] for Female, and [201, 300] for Male) randomization methods (using minimization or using rand list, with or without dynamic blocking), type of blocks (size and kit ratio used in each block) with the count of each block type, layout of the list (column headers and content of each column), file naming convention, and a delimiter to use in the generated file.

An action interpreter can interpret event actions information in the specification. For example, an event action can include information such as what drug dispensing may occur within the context of each patient visit. A drug supply interpreter can interpret the drug supply information in the specification. For example, the drug supply information can include supply network, shipping rules, dispensing kits info, etc. A manufacturing interpreter may interpret the various production steps and intermediate manufacturing lead time to transform the investigational substance into kits dispensable to patients. An enrollment interpreter may interpret the rate at which patients will enter the study at various levels (e.g. study-level, region-level, etc.). The list of specialized interpreters is not exhaustive. Additional specialized interpreters can be added when needed.

In some situations, the master interpreter 602 can be an entry point into the specification interpreter 600. For example, the master interpreter 602 can receive a specification and divide the interpretation into different sections or themes. The master interpreter 602 can then invoke an appropriate specialized interpreter 604 to analyze each section or theme. Each specialized interpreter 604 can invoke one or more information extractors 606, as needed, to extract information from each section or theme of the specification, such as matrices and definitions. Each information extractor 606 can invoke one or more specialized NLP tools 608, as needed. Each specialized NLP tool 608 can then invoke one or more core NLP libraries 610 to assist processing each section or theme of the specification.

The output result of a core NLP library 610 can be returned to the specialized NLP tool 608 that invokes the core NLP library 610. The output result of a specialized NLP tool 608 can be returned to the information extractor 606 that invokes the specialized NLP library 610. The output result of an information extractor 606 can be returned to the specialized interpreter 604 that invokes the information extractor 606. The output result of a specialized interpreter 604 can be returned to the master interpreter 602 that invokes the specialized interpreter 604.

In some embodiments, the output of the specification interpreter 600 can include basic data structures and driving logic. The basic data structures and driving logic can include logic containers and other necessary or related structures. For example, the data structures may hold planned patient visit and dosing information, data driving information (e.g. BMI for use in dosing), data related to supply chain organization, supply shipments and/or patient kits, or any other data which is desired to be collected or acted upon during the trial. Where necessary, logic containers can hold specific processing instructions for the forecasting system in order to comply with the specification.

FIG. 7 illustrates an example graphical display 700 of a randomized patient list that may be generated and output by the validation system of FIG. 1 after validating (or attempting to validate) a randomized patient list. The display may include an ordered plurality of coded adjacent squares or other shapes, with each shape corresponding to an individual patient or kit. The shapes may be ordered the same as the patient list. The coding of squares may be color coding, pattern coding, or other coding that enables visual distinction of one square type from another. In the embodiment illustrated, squares are color-coded, with patients assigned to the active drug arm a first color and patients assigned to the placebo arm a second color. Accordingly, through manual inspection, a user can quickly assess the distribution of patients across the trial, or across a portion of the trial to better understand the numerical and statistical validation information generated by the validation system.

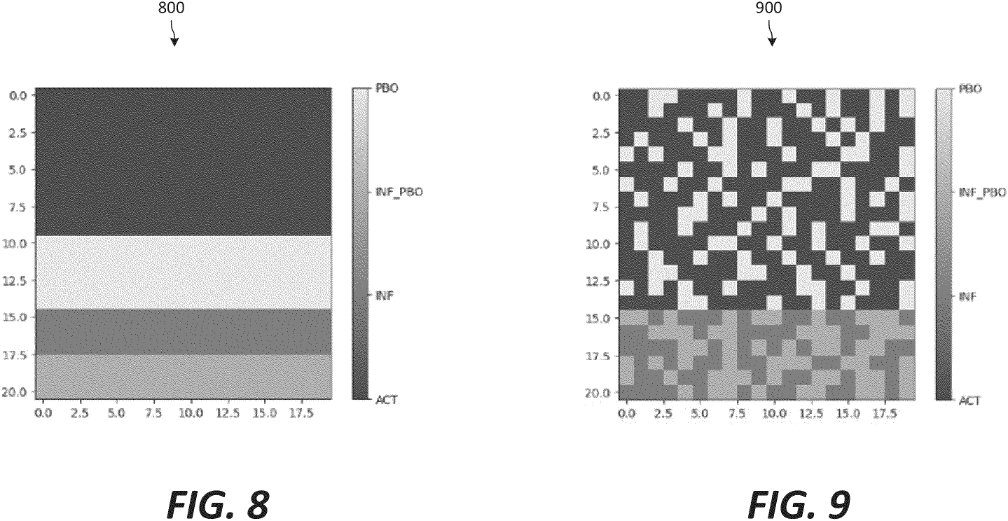

FIGS. 8 and 9 illustrate example graphical displays 800, 900 of a randomized kit list that may be generated and output by the validation system of FIG. 1 after validating (or attempting to validate) a randomized kit list. The displays 800, 900 are respective of the same kit list, but are differently ordered. The first display 800 is ordered by kit sequence (i.e., grouping all kits of the same type), and the second is ordered by kit number. Like the patient list display of FIG. 7, the kit list displays 800, 900 may be color-coded, pattern coded, or otherwise coded to enable visual distinction of one square type from another. In the embodiment illustrated, squares are color-coded, with each of four kit types having its own respective color. As a result, in the display 800, a user can quickly visually confirm that the relative quantities of different kit types is appropriate. In the display 900, a user can quickly assess the distribution of kits across the trial, or across a portion of the trial, including the lengths of runs, sequences of runs, etc., to better understand the numerical and statistical validation information generated by the validation system.

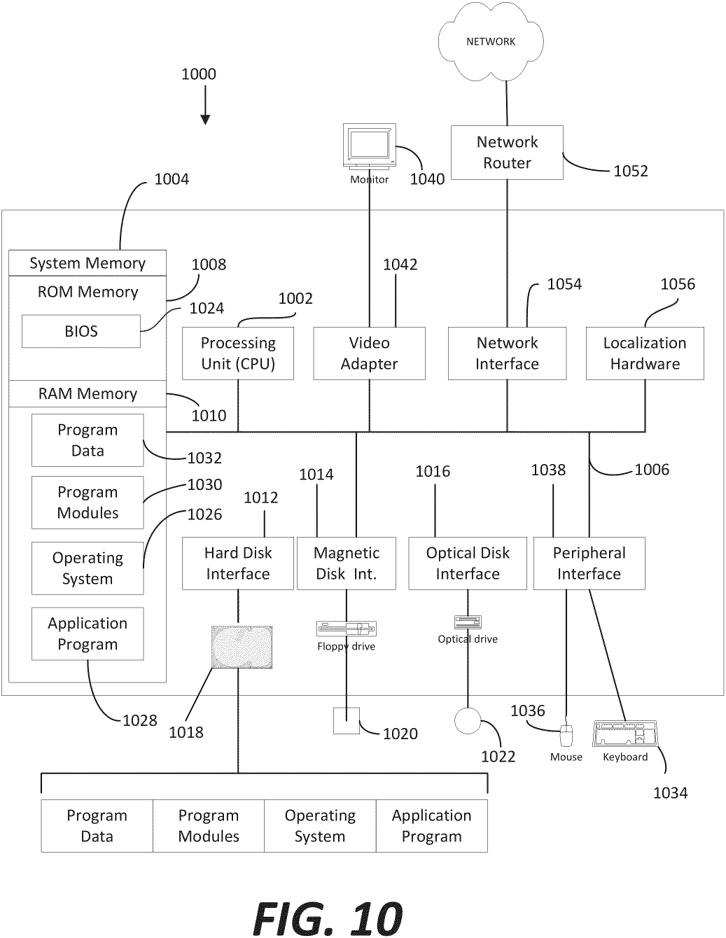

FIG. 10 is a diagrammatic view of an example embodiment of a user computing environment that includes a general purpose computing system environment 1000, such as a desktop computer, laptop, smartphone, tablet, or any other such device having the ability to execute instructions, such as those stored within a non-transient, computer-readable medium. Furthermore, while described and illustrated in the context of a single computing system 1000, those skilled in the art will also appreciate that the various tasks described hereinafter may be practiced in a distributed environment having multiple computing systems 1000 linked via a local or wide-area network in which the executable instructions may be associated with and/or executed by one or more of multiple computing systems 1000.

In its most basic configuration, computing system environment 1000 typically includes at least one processing unit 1002 and at least one memory 1004, which may be linked via a bus 1006. Depending on the exact configuration and type of computing system environment, memory 1004 may be volatile (such as RAM 1010), non-volatile (such as ROM 1008, flash memory, etc.) or some combination of the two. Computing system environment 1000 may have additional features and/or functionality. For example, computing system environment 1000 may also include additional storage (removable and/or non-removable) including, but not limited to, magnetic or optical disks, tape drives and/or flash drives. Such additional memory devices may be made accessible to the computing system environment 1000 by means of, for example, a hard disk drive interface 1012, a magnetic disk drive interface 1014, and/or an optical disk drive interface 1016. As will be understood, these devices, which would be linked to the system bus 1006, respectively, allow for reading from and writing to a hard disk 1018, reading from or writing to a removable magnetic disk 1020, and/or for reading from or writing to a removable optical disk 1022, such as a CD/DVD ROM or other optical media. The drive interfaces and their associated computer-readable media allow for the nonvolatile storage of computer readable instructions, data structures, program modules and other data for the computing system environment 1000. Those skilled in the art will further appreciate that other types of computer readable media that can store data may be used for this same purpose. Examples of such media devices include, but are not limited to, magnetic cassettes, flash memory cards, digital videodisks, Bernoulli cartridges, random access memories, nano-drives, memory sticks, other read/write and/or read-only memories and/or any other method or technology for storage of information such as computer readable instructions, data structures, program modules or other data. Any such computer storage media may be part of computing system environment 1000.

A number of program modules may be stored in one or more of the memory/media devices. For example, a basic input/output system (BIOS) 1024, containing the basic routines that help to transfer information between elements within the computing system environment 1000, such as during start-up, may be stored in ROM 1008. Similarly, RAM 1010, hard drive 1018, and/or peripheral memory devices may be used to store computer executable instructions comprising an operating system 1026, one or more applications programs 1028 (which may include the functionality of the randomized list generation and validation system 102 and/or the RTSM system 104 of FIG. 1, for example), other program modules 1030, and/or program data 1022. Still further, computer-executable instructions may be downloaded to the computing environment 1000 as needed, for example, via a network connection.

An end-user may enter commands and information into the computing system environment 1000 through input devices such as a keyboard 1034 and/or a pointing device 1036. While not illustrated, other input devices may include a microphone, a joystick, a game pad, a scanner, etc. These and other input devices would typically be connected to the processing unit 1002 by means of a peripheral interface 1038 which, in turn, would be coupled to bus 1006. Input devices may be directly or indirectly connected to processor 1002 via interfaces such as, for example, a parallel port, game port, firewire, or a universal serial bus (USB). To view information from the computing system environment 1000, a monitor 1040 or other type of display device may also be connected to bus 1006 via an interface, such as via video adapter 1032. In addition to the monitor 1040, the computing system environment 1000 may also include other peripheral output devices, not shown, such as speakers and printers.

The computing system environment 1000 may also utilize logical connections to one or more computing system environments. Communications between the computing system environment 1000 and the remote computing system environment may be exchanged via a further processing device, such a network router 1052, that is responsible for network routing. Communications with the network router 1052 may be performed via a network interface component 1054. Thus, within such a networked environment, e.g., the Internet, World Wide Web, LAN, or other like type of wired or wireless net work, it will be appreciated that program modules depicted relative to the computing system environment 1000, or portions thereof, may be stored in the memory storage device(s) of the computing system environment 1000.

The computing system environment 1000 may also include localization hardware 1056 for determining a location of the computing system environment 1000. In embodiments, the localization hardware 1056 may include, for example only, a GPS antenna, an RFID chip or reader, a WiFi antenna, or other computing hardware that may be used to capture or transmit signals that may be used to determine the location of the computing system environment 1000.

While this disclosure has described certain embodiments, it will be understood that the claims are not intended to be limited to these embodiments except as explicitly recited in the claims. On the contrary, the instant disclosure is intended to cover alternatives, modifications and equivalents, which may be included within the spirit and scope of the disclosure. Furthermore, in the detailed description of the present disclosure, numerous specific details are set forth in order to provide a thorough understanding of the disclosed embodiments. However, it will be obvious to one of ordinary skill in the art that systems and methods consistent with this disclosure may be practiced without these specific details. In other instances, well known methods, procedures, components, and circuits have not been described in detail as not to unnecessarily obscure various aspects of the present disclosure.

Some portions of the detailed descriptions of this disclosure have been presented in terms of procedures, logic blocks, processing, and other symbolic representations of operations on data bits within a computer or digital system memory. These descriptions and representations are the means used by those skilled in the data processing arts to most effectively convey the substance of their work to others skilled in the art. A procedure, logic block, process, etc., is herein, and generally, conceived to be a self-consistent sequence of steps or instructions leading to a desired result. The steps are those requiring physical manipulations of physical quantities. Usually, though not necessarily, these physical manipulations take the form of electrical or magnetic data capable of being stored, transferred, combined, compared, and otherwise manipulated in a computer system or similar electronic computing device. For reasons of convenience, and with reference to common usage, such data is referred to as bits, values, elements, symbols, characters, terms, numbers, or the like, with reference to various presently disclosed embodiments. It should be borne in mind, however, that these terms are to be interpreted as referencing physical manipulations and quantities and are merely convenient labels that should be interpreted further in view of terms commonly used in the art. Unless specifically stated otherwise, as apparent from the discussion herein, it is understood that throughout discussions of the present embodiment, discussions utilizing terms such as “determining” or “outputting” or “transmitting” or “recording” or “locating” or “storing” or “displaying” or “receiving” or “recognizing” or “utilizing” or “generating” or “providing” or “accessing” or “checking” or “notifying” or “delivering” or the like, refer to the action and processes of a computer system, or similar electronic computing device, that manipulates and transforms data. The data is represented as physical (electronic) quantities within the computer system’s registers and memories and is transformed into other data similarly represented as physical quantities within the computer system memories or registers, or other such information storage, transmission, or display devices as described herein or otherwise understood to one of ordinary skill in the art.

What is claimed is:

1. A method for managing a clinical trial for a drug, the method comprising:
receiving, by a server from a user, one or more electronic files that contain parameters of a clinical trial;
performing, by the server, natural language processing on the one or more electronic files to determine the parameters;
generating a randomized patient list based on the determined parameters;
validating, by the server, the randomized patient list by:
verifying that the patient list includes a balance or spread above a balance or spread threshold; and
verifying that the list has a randomness above a predetermined randomness threshold; and
configuring, by the server, an RTSM system according to the validated randomized patient list.

2. The method of claim 1, further comprising:
distributing and dispensing kits of the drug to patients according to the configured RTSM system.

3. The method of claim 1, further comprising:
verifying that the patient list contains valid content by one or more of:
validating a total quantity of rows and a total quantity of blocks in the patient list;
validating a quantity of rows and a quantity of blocks for one or more treatment arms;
validating a quantity of rows and a quantity of blocks for one or more cohort and strata combinations; or
validating a size and quantity of a plurality of block types.

4. The method of claim 1, wherein verifying that the patient list includes a balance or spread above a predetermined threshold comprises:
computing a plurality of simulations of patient enrollment; and
computing the balance or spread according to the simulations.

5. The method of claim 4, wherein computing a plurality of simulations of patient enrollment comprises assuming an equal likelihood of patient enrollment in each of a plurality of strata or cohorts of the trial.

6. The method of claim 1, wherein verifying that the list has a randomness above a predetermined randomness threshold comprises:
determining a block pattern distribution within the list;
calculating a probability of obtaining a distribution of block patterns at least as extreme as the observed block pattern distribution; and
comparing the probability to the predetermined randomness threshold.

7. A method for managing a clinical trial for a drug, the method comprising:
receiving, by a server from a user, one or more electronic files that contain parameters of a clinical trial;
performing, by the server, natural language processing on the one or more electronic files to determine the parameters;
generating a randomized kit list based on the determined parameters;
validating, by the server, the randomized kit list by:
verifying that the kit list has a randomness above a predetermined randomness threshold; and
configuring, by the server, an RTSM system according to the validated randomized kit list.

8. The method of claim 7, further comprising:
distributing and dispensing kits of the drug to patients according to the configured RTSM system.

9. The method of claim 7, wherein validating the randomized kit list comprises verifying that the kit list contains valid content by one or more of:
validating a total quantity of rows and a total quantity of blocks in the kit list; or
validating a quantity of rows and a quantity of blocks for one or more kit types.

10. The method of claim 7, wherein verifying that the list has a randomness above a predetermined randomness threshold comprises:
for a non-blocked list, determining that a probability of a distribution of run lengths in the kit list exceeds the threshold; or
for a blocked list:
determining a block pattern distribution within the list;
calculating a probability of obtaining a distribution of block patterns at least as extreme as the observed block pattern distribution; and
comparing the probability to the predetermined randomness threshold.

11. A method for managing a clinical trial for a drug, the method comprising:
receiving, by a server from a user, one or more electronic files that contain parameters of a clinical trial;
performing, by the server, natural language processing on the one or more electronic files to determine the parameters;
generating a randomized patient list and a randomized kit list based on the determined parameters;
validating, by the server, the randomized patient list by:
verifying that the patient list includes a balance or spread above a balance or spread threshold; and
verifying that the list has a randomness above a predetermined patient list randomness threshold;
validating, by the server, the randomized kit list by:
verifying that the kit list has a randomness above a predetermined kit list randomness threshold; and
configuring, by the server, an RTSM system according to the validated randomized patient list and the validated randomized kit list.

12. The method of claim 11, further comprising:
distributing and dispensing kits of the drug to patients according to the configured RTSM system.

13. The method of claim 11, wherein validating the randomized patient list comprises verifying that the patient list contains valid content by one or more of:
validating a total quantity of rows and a total quantity of blocks in the patient list;
validating a quantity of rows and a quantity of blocks for one or more treatment arms;
validating a quantity of rows and a quantity of blocks for one or more cohort and strata combinations; or
validating a size and quantity of a plurality of block types.

14. The method of claim 11, wherein verifying that the patient list includes a balance or spread above a predetermined threshold comprises:
computing a plurality of simulations of patient enrollment; and
computing the balance or spread according to the simulations.

15. The method of claim 14, wherein computing a plurality of simulations of patient enrollment comprises assuming an equal likelihood of patient enrollment in each of a plurality of strata or cohorts of the trial.

16. The method of claim 11, wherein verifying that the patient list has a randomness above a predetermined patient list randomness threshold comprises:
determining a block pattern distribution within the patient list;
calculating a probability of obtaining a distribution of block patterns at least as extreme as the observed block pattern distribution; and
comparing the probability to the predetermined randomness threshold.

17. The method of claim 11, wherein validating the kit list comprises verifying that the kit list includes valid content by one or more of:
validating a total quantity of rows and a total quantity of blocks based on the one or more electronic files; or
validating a quantity of rows and a quantity of blocks for one or more kit types.

18. The method of claim 11, wherein verifying that the kit list has a randomness above a predetermined randomness threshold comprises:
for a non-blocked kit list, determining that a probability of a distribution of run lengths in the kit list exceeds the threshold; or
for a blocked kit list:
determining a block pattern distribution within the kit list;
calculating a probability of obtaining a distribution of block patterns at least as extreme as the observed block pattern distribution; and
comparing the probability to the predetermined randomness threshold.

19. The method of claim 11, wherein the randomized patient list is a second randomized patient list and the randomized kit list is a second randomized kit list, the method further comprising:
generating a first randomized patient list and a first randomized kit list based on the determined parameters;
attempting to validate, by the server, the first randomized patient list;
attempting to validate, by the server, the randomized kit list;
determining, by the server, that the randomized patient list or the randomized kit list is not valid and, in response:
outputting, by the server, an error; and
determining, by the server, the second randomized patient list and the second randomized kit list.

20. The method of claim 11, further comprising generating and outputting a report of the validation of the randomized patient list and the randomized kit list, the report comprising a graphical representation of one or more of the randomized patient list or the randomized kit list.
